(19) United States
(12) Patent Application Publication
(10) Pub. No.: US 2011/0027341 A1
(43) Pub. Date: Feb. 3, 2011

(54) DEVICE FOR IN SITU PRODUCTION AND TOPICAL ADMINISTRATION OF ALLICIN

(75) Inventors: David Mirelman, Ramat Efal (IL); Aharon Rabinkov, Rehobot (IL)

Correspondence Address: Browdy and Neimark, PLLC, 1625 K Street, N.W., Suite 1100, Washington, DC, 20006, US

(73) Assignee: YEDA RESEARCH AND DEVELOPMENT CO., LTD. at the Weizmann Institute of Science, Rehovot (IL)

(21) Appl. No.: 12/936,022

(22) PCT Filed: Mar. 26, 2009
(86) PCT No.: PCT/IL2009/000350

§ 371 Date: Oct. 1, 2010

Related U.S. Application Data
(60) Provisional application No. 61/041,452, filed on Apr. 1, 2008.

(51) Int. Cl. A61F 6/08; A61K 38/51; A61L 15/44; A61L 15/58; A61P 31/10; A61P 31/04

(52) U.S. Cl. 424/430; 424/94.5; 424/447

(57) ABSTRACT

The present invention relates to a drug delivery device that is useful for topical treatment of various infections such as skin and nail, or vaginal infections. More specifically, the invention provides a device for topical administration of allicin to an infection site, comprising either one solid carrier or two adjacent solid carriers, dry alliin and dry alliinase, wherein either a mixture of said dry alliin and dry alliinase is contained within said one solid carrier or dry alliin and dry alliinase are each separately contained within each one of said two adjacent solid carriers, whereby in contact with the infection site and a wetting agent, the alliinase acts on the alliin and allicin is produced in situ and administered to the infection site

### FIELD OF THE INVENTION

The present invention relates to a drug delivery device, in particular to a device that produces allicin in situ and administers it to an infection site for topical treatment of various infections such as skin and nail, or vaginal infections.

### BACKGROUND ART

### Nail Fungal Infections

Fungal infections of nails account for about half of all the nail disorders and are estimated to occur in over 10% of the population. An infection by a nail fungus (onychomycosis) occurs when a dermatophyte infects one or more of the nails, and usually begins as a white or yellow spot under the tip of the nail. As the nail fungus spreads deeper into the nail, it may cause it to discolor, thicken and develop crumbling edges, an unsightly and potentially painful problem. These infections usually develop on nails continually exposed to warm and moist environments such as sweaty shoes or shower floors, may be difficult to treat, and may recur.

Some medications may help clear up nail fungus, including oral medications such as itraconazole (Sporanox®), fluconazole (Diflucan®) and terbinafine (Lamisil®) that are available under prescription. However, antifungal drugs may cause side effects ranging from skin rashes to liver damage and therefore, may not be recommended for people with liver disease or congestive heart failure. Such medications should be taken for 6-12 weeks, but the end result of treatment cannot be seen until the nail grows back completely, thus, it may take 4-12 months to eliminate an infection, while recurrent infections are possible.

An alternative treatment is based on the use of antifungal lacquers and topical medications. For moderate infections of nail fungus, the Food and Drug Administration has approved a topical antifungal lacquer called ciclopirox (Penlac®), which is painted onto the infected nails and surrounding skin once a day; and once a week, the piled-on layers are cleaned with alcohol and a fresh application is started. Daily use of Penlac® for up to one year or more has been shown to help clear nail fungal infections; however, it cured the infections in less than 10% of people using it. Another topical antifungal medication is econazole nitrate (Spectazole). In some cases, it is advised to use these creams with an over-the-counter lotion containing urea to help speed up absorption. Topical medications usually do not provide a cure but may be used in conjunction with oral medications.

U.S. Pat. No. 7,074,392 describes a sustained release therapeutic nail varnish composition comprising an antifungal agent, a keratolytic agent, e.g., urea, a humectant, water and a liquid nail lacquer component comprising a polymeric film forming agent and a volatile solvent.

U.S. Pat. No. 7,094,422 describes a topical drug delivery system which comprises an antifungal agent, at least one dermal penetration enhancer, e.g., octyl salicylate, and a volatile liquid.

U.S. Pat. No. 6,727,401 describes a device for treating antifungal infections of toenails and fingernails made up of an occlusive backing layer and a pressure sensitive adhesive matrix layer in which is uniformly dispersed an effective amount of an antifungal agent and, optionally, a chemical enhancer. The matrix layer has a first surface adhering to the backing layer and a second surface adapted to be in diffusional contact with the infected nail and surrounding skin area. The device is configured, when applied, to cover and adhere to the nail and surrounding skin areas for an extended period of time without causing irritation to the skin or inhibiting normal physical activity while providing a continuous delivery of antifungal agent to the infected area.

### Vaginal Infections

Vaginitis is a medical term used to describe various conditions associated with infection or inflammation of the vagina. The most common types of vaginitis associated with infection are candida or “yeast” infection, bacterial vaginosis, trichomonas vaginitis, chlamydia vaginitis and viral vaginitis. Although each of these types of vaginal infections can have different symptoms, it is not always easy to figure out the type of the specific vaginitis, and diagnosis can be complicated even for an experienced clinician, partially due to the fact that more than one type of vaginitis can be present at the same time.

Yeast infections are a common cause of vaginitis, producing a thick, white vaginal discharge with the consistency of cottage cheese. Although the discharge can be somewhat watery, it is odorless. Yeast infections usually cause the vagina and vulva to be very itchy and red. An antibiotic taken for a urinary tract infection can kill “friendly” bacteria that normally keep the yeast in balance; as a result, the yeast overgrows and causes the infection.

Bacterial vaginosis is a condition in which the normal balance of the vaginal bacteria is disrupted and replaced by an overgrowth of certain bacteria. Bacterial vaginitis results in a vaginal discharge that is usually thin and milky and is sometimes described as having a “fishy” odor, which may become more noticeable after intercourse. Treatment is usually with antibiotics.

Trichomonas vaginitis is caused by protozoa and can be transmitted through sexual intercourse. When this organism infects the vagina it can cause a frothy, greenish-yellow discharge that often has a foul smell. The symptoms include itching and soreness of the vagina and vulva, and burning during urination, and may be worse after a menstrual period.

Chlamydia vaginitis is a sexually transmitted disease mostly common in young women under the age of 30, and in most cases has no symptoms. A vaginal discharge is not always present, although light bleeding may appear especially after intercourse.

Viral vaginitis can be caused by herpes simplex virus transmitted through sexual intercourse. The primary symptom of herpes vaginitis is pain associated with lesions or sores, which can be seen on the vulva or the vagina during a gynecologic examination.

Currently there is no single drug effective against all the above-mentioned possible infecting pathogens, and none of the drugs available has antiviral effect. Yeast infections are most often treated with an antifungal of the type of nocodazole that is available as a cream and in a suppository form, or in pills under prescription, wherein butoconazole (Femstat®, Mycelex, Gynazole cream) intravaginally for 3 days is the drug of choice. However, there are many fungal species that are resistant to various topical treatments. In particular, Torulopsis (Candida) glabrata as well as Candida albicans and Saccharomyces cerevisiae are more resistant (in vitro) to clotrimazole and ketoconazole; and C. krusei strains have shown resistance to nystatin and flucytosine. Other therapies use terconazole (Terazole®), which is somewhat more effective than fluconazole (Diflucan®) for many species. Boric acid vaginal suppositories at 600 mg/day for 10 days were found to be 80% effective for C. glabrata, and tea tree essential oil has been shown to be effective against yeast in concentrations of 0.5-2%.

The most common medicines prescribed for bacterial and trichomonas infections are metronidazole (Flagyl) and clindamycin (Cleocin), and a variety of vaginal suppositories are currently available. For treating vaginal mycosis, clotrimazole (Canesten®) and natamycin/pimaricin (Pimafucin®) are most commonly used. For fungal and protozoan infections, metronidazole (Klion-D) is currently available as pills and as vaginal creams or gels.

### Allicin

Allicin is the diallyl thiosulfinate molecule produced upon crushing of garlic cloves and is the substance responsible for the typical smell of freshly crushed garlic cloves. In particular, it is produced by the catalytic action of the garlic enzyme alliinase (Rabinkov et al., 1994) on the substrate alliin, both present in separate compartments in the garlic clove.

Allicin has superb antifungal (Ankri and Mirelman, 1999; Schadkchan et al., 2004), antibacterial, antiviral and antiprotozoal, including anti-Trichomonas, activity. It has antimicrobial lethal doses that are in the micromolar concentrations and, in fact, there are almost no known microorganisms resistant to its lethal action. Allicin's mode of action is different from that of other antibiotics or antimicrobials in that it very rapidly penetrates through microbial cell membranes and reacts by thiolation to modify free thiol groups present on a variety of proteins, including numerous essential metabolic enzymes (Mixon et al., 2000; Prager-Khoutorsky et al., 2007).

However, allicin is a very reactive and chemically unstable molecule, which is sensitive to heat and has a short shelf life, e.g., when kept at cold temperatures and at a pH of around 6, it degrades at a rate of 8%/month. These are the main reasons why there are no allicin-containing antifungal products available, which guarantee the availability of known amounts of allicin for therapeutic uses.

Purified alliinase has been shown to rapidly generate measurable quantities of allicin, identical to the natural product, from known amounts of synthetic substrate alliin. U.S. Pat. No. 6,689,588 describes chemically immobilized garlic alliinase comprising garlic alliinase chemically immobilized by covalent binding to a carrier selected from organic natural and synthetic polymers or inorganic carriers. In this form, alliinase retains its activity and can be used for the continuous preparation of allicin from alliin without losing its biological activity.

### SUMMARY OF INVENTION

In one aspect, the present invention relates to a device for topical administration of allicin to an infection site, comprising either one solid carrier or two adjacent solid carriers, dry alliin and dry alliinase, wherein either a mixture of said dry alliin and dry alliinase is contained within said one solid carrier or dry alliin and dry alliinase are each separately contained within each one of said two adjacent solid carriers, whereby in contact with the infection site and a wetting agent, the alliinase acts on the alliin and allicin is produced in situ and administered to the infection site.

The device of the present invention is useful for treatment of bacterial and fungal skin infections, fungal toenail and fingernail infections, preferably onychomycosis, or bacterial and fungal vaginal infections.

In another aspect, the present invention relates to a kit for topical administration of allicin to an infection site comprising the device defined hereinabove and optionally a container comprising the wetting agent.

In a further aspect, the present invention provides a method for treating topically an infection site comprising applying to the infection site the device defined hereinabove, optionally followed by applying a suitable amount of a wetting agent to said device, thus producing in situ allicin and continuously delivering said allicin to the infection site to thereby treat the infection.

### BRIEF DESCRIPTION OF DRAWINGS

FIG. 1 shows a schematic prototype of a device according to the present invention, for topical administration of allicin to treat a fungal nail infection. The prototype shown comprises a first dry matrix onto which a known amount of alliinase solution was adsorbed and dried, placed on top of a second dry matrix onto which a known amount of alliin solution was absorbed and dried, wherein said two adjacent matrices are placed in an adhesive antiseptic bandage on top of which a small bag containing an aqueous buffer is placed, and said adhesive antiseptic bandage can be placed on an infected nail. The bag containing the liquid buffer is made of a material that is impermeable to water; however, designed to rupture and spill its content upon application of some pressure. Once the adhesive bandage is well placed on the infected nail and secured around the finger, a small pressure can be applied with a finger on the said bag, causing the bag to rupture and allow the liquid to wet the dry matrices below it.

FIGS. 2A-2B show a prototype of a device according to the present invention, configured as a vaginal insertable device, i.e., a vaginal tampon, containing a prodrug preparation, i.e., a mixture of dry alliin and dry alliinase, inserted into the fissure of the cotton (2A); and such a vaginal tampon inserted into a tube simulating a vaginal cavity and containing liquid medium with either bacteria or yeast (2B).

FIGS. 3A-3b show the antifungal activity of the two-filter delivery system. FIG. 3A shows Petri dish plates with Trichoderma hyphae (6 hours growth at 28° C. after seeding of 5×10⁶ spores) in which glass fiber filters containing both alliin and alliinase (top left), only alliin (top center) or only alliinase (top right) were placed vs. Petri dish plates without any fungal growth (bottom left) or with Trichoderma hyphae incubated for 24 hours (bottom right) as negative and positive controls, respectively. FIG. 3B shows a similar experiment, in which glass fiber filters containing both alliin and alliinase (left), only alliin (center) or only alliinase (right) were placed in the Petri dish plates after 24 hours incubation and the plates were then incubated for additional 16 hours.

FIGS. 4A-4C show the effect of pure allicin and of mixtures of dry alliin and dry alliinase on various bacteria, in particular, group B streptococci (10⁸ bacteria) seeded on blood agar plate (4A), vancomycin-intermediate Staphylococcus aureus (VISA) seeded on regular nutrient agar plate (4B) and methicillin-resistant Staphylococcus aureus (MRSA) seeded on regular nutrient agar plate (4C), following overnight incubation at 37° C. Al/Al—P 70 and Al/Al—P 100 represent dry glass fiber filters containing a mixture of dry alliin (70 or 100 μg, respectively) and dry alliinase (2 units), which were placed on the seeded agar plate and then wetted with 100 μl water; and allicin (5, 20 or 30 μg) represents dry glass fiber filters on which the indicated amount of pure allicin was dripped just before placing on the seeded agar. Control—a dry glass fiber filter.

FIGS. 5A-5B show the effect of pure allicin and of a mixture of dry alliin and dry alliinase on Candida albicans (5A) and Candida glabrata (5B) seeded on agar plates, following overnight incubation at 30° C. FIG. 5A shows the effect of pure allicin (20 μg/ml) on Candida albicans, wherein in the left side of the plate, an aliquot taken from a cultivation medium of the yeast that was treated with the indicated concentration of allicin was seeded, and in the right side, an aliquot taken from an identical cultivation medium of the yeast that was not treated with allicin was seeded. FIG. 5B shows the effect of pure allicin (30 μg/ml) and of a mixture of dry alliin (100 μg) and dry alliinase (2 units) (Al/Al—P 100) on Candida glabarta. Control—a dry glass fiber filter.

### MODES FOR CARRYING OUT THE INVENTION

In preliminary experiments conducted by the inventors of the present invention, a method for isolation of the natural substrate alliin from garlic cloves has been developed, and the enzyme alliinase has been purified and stabilized in a dry form. Mixing of various ratios of dry alliin with dry alliinase, both in a powder form, were shown to yield different amounts of allicin upon wetting of the dry mixtures. Furthermore, both components in their dry form at room temperature preserved their potential for producing allicin for several months.

The present invention thus relates to a device for topical administration of allicin to an infection site, comprising either one solid carrier or two adjacent solid carriers, dry alliin and dry alliinase, wherein either a mixture of said dry alliin and dry alliinase is contained within said one solid carrier or dry alliin and dry alliinase are each separately contained within each one of said two adjacent solid carriers, whereby in contact with the infection site and a wetting agent, alliinase acts on the alliin and allicin is produced in situ and administered to the infection site.

The alliin used in the device of the present invention may be isolated from garlic clove pretreated in a microwave oven to inactivate the alliinase enzyme, which is present in the same material using extraction with ethanol. The alliinase used in the device of the present invention may be either natural alliinase, which may be isolated from garlic cloves by any conventional method including, e.g., precipitation with polyethylene glycol, and ion-exchange chromatography; or recombinant alliinase such as disclosed in International Publication No. WO 94/08614. The aforesaid two components, each prepared in an aqueous solution, may then be dried using any suitable drying method such as lyophilization, and used for the preparation of various mixtures thereof containing different amounts and ratios of dry alliin and dry alliinase, referred herein also as “prodrug preparations”. Alternatively, predetermined amounts of alliin or alliinase solutions are adsorbed, each one separately, onto dry solid carriers or matrices that are then dried by, e.g., lyophilization.

The amount of alliin in the dry powder obtained from the alliin solution prepared is determined by the amount of allicin that can be produced from that amount of dry powder following incubation at room temperature for 30 min with an excess amount of purified alliinase, and using purified allicin quantitatively determined by HPLC analysis and stored at −80° C. as a standard solution. The amount of alliinase in the dry powder obtained from the alliinase solution prepared is determined in a similar manner, based on the determined activity of that alliinase, wherein one unit of alliinase activity is defined as the amount of enzyme converting alliin into pyruvic acid and allicin at a rate of 1 μmol/min (Miron et al., 2002 and 2006). The amounts of alliin and alliinase, either contained separately within two solid carriers or as a prodrug preparation within a single solid carrier, required in order to yield a certain amount of allicin upon wetting of these carriers, is then determined using the calibration process described hereinabove. Thus, the advantage of the prodrug preparation is in its potential to produce controlled amounts of allicin as an effective broad spectrum anti-microbial agent, using certain amounts of each one of the two components, i.e., alliin and alliinase, predetermined as described above.

In preliminary experiments conducted by the inventors of the present invention, various amounts of alliin (100-200 μl from a solution of 50 mg/ml in water) and alliinase (100-200 μl from a solution of 120 enzyme units/ml in PBS pH 7.2 containing 5% mannitol) were adsorbed on different types of matrices, e.g., glass fiber filters, and upon wetting of the two matrices, when placed one on top of the other, using an aqueous buffered solution, different amounts of allicin were produced. For example, glass fiber filter matrices containing 2.5-10 mg of alliin together with glass fiber filter matrices each containing 14 units of dry alliinase yielded between 0.5-1.0 mg allicin following wetting of the two matrices and incubation at room temperature for 30 minutes. No allicin was produced prior to the wetting of the dry matrices by the introduction of the aqueous fluid nor after incubating each matrix wetted in separate. The potential of the dry glass fiber filter matrices, as long as kept dry, to produce the reproducible amounts of allicin upon wetting did not diminish more than 5% after 2 months.

The carrier, also referred herein as the matrix, according to the present invention, may be any suitable absorbent carrier or matrix such as, without being limited to, glass fiber filter, cotton, gauze and a polysaccharide-based polymer absorbent material, e.g., starch, cellulose, etc. In preferred embodiments, the carrier or matrix is glass fiber filter or cotton.

The term “two adjacent solid carriers” as used herein refers to any two solid carriers as defined herein, placed one on top of the other to bring them into physical contact.

The wetting agent according to the present invention may be any suitable aqueous solution with a pH range of 6-7.5, preferably around 7.2.

In one embodiment, the wetting agent is a buffer that may be of different compositions. Non-limiting examples of such buffers include citrate buffer or phosphate-buffered saline (PBS) of pH in the range of 6-7.5, preferably around 7.2.

In certain cases, preferably wherein the device is used for treatment of bacterial or fungal skin infections, or fungal nail infections, the wetting agent buffer may further contain a permeability enhancer or adjuvant. The permeability enhancer can be any suitable skin or nail keratin penetration enhancer, preferably urea.

In another embodiment, the wetting agent is a bodily fluid such as, without limitation, vaginal lubrication or saliva.

In one embodiment, the device of the present invention comprises one solid carrier containing the mixture of alliin and alliinase, wherein said carrier is placed in a bandage. In another embodiment, the device comprises two adjacent solid carriers, each containing either the alliin or the alliinase, wherein said carriers are placed one on top of the other in a bandage. In a preferred embodiment, the device of the present invention comprises two adjacent solid carriers.

In one preferred embodiment, the bandage is an adhesive band, e.g., a “Band-Aid Type” bandage.

In one preferred embodiment, the device of the present invention comprises a hole on the top of the bandage for placing a small drop bottle containing the wetting agent. According to another preferred embodiment, the device comprises a small bag containing the wetting agent on top of said one solid carrier or two adjacent solid carriers. The bag containing the wetting agent is most preferably made of a water-impermeable material designed for rupturing under a moderate, i.e., gentle, pressure, thus spilling the wetting agent on top of said one solid carrier or two adjacent solid carriers. It should be understood that the bag containing the wetting agent might be ruptured or punctured without the aid of a mechanical device, e.g., by gently pressing on said bag with a finger.

Example 4 in the Example section hereinafter shows the antifungal activity of allicin produced in situ following wetting of two adjacent solid carriers to each of which either alliin or alliinase were adsorbed, illustrated as inhibition of Trichoderma hyphae growth.

Thus, in on embodiment, the device of the present invention, when configured either as a single solid carrier or two adjacent solid carriers placed one on top of the other, in a bandage, is used for treatment of bacterial or fungal infections of the skin. Since allicin may be irritant to the skin, the amounts of alliin and alliinase absorbed to the carriers should be designed so that the amount of allicin produced upon wetting of the carriers and delivered to the skin is very small and non-irritating.

In another embodiment, the device of the present invention, when configured either as a single solid carrier or two adjacent solid carriers placed one on top of the other, in a bandage, is used for treatment of fungal infections of toenails and fingernails. In preferred embodiments, fungal toenail infections, most preferably onychomycosis, are treated. For treatment of onychomycosis in toenails, the device of the invention may be configured so as to cover and adhere to the nail and surrounding skin areas while providing a continuous delivery of allicin to the infected area.

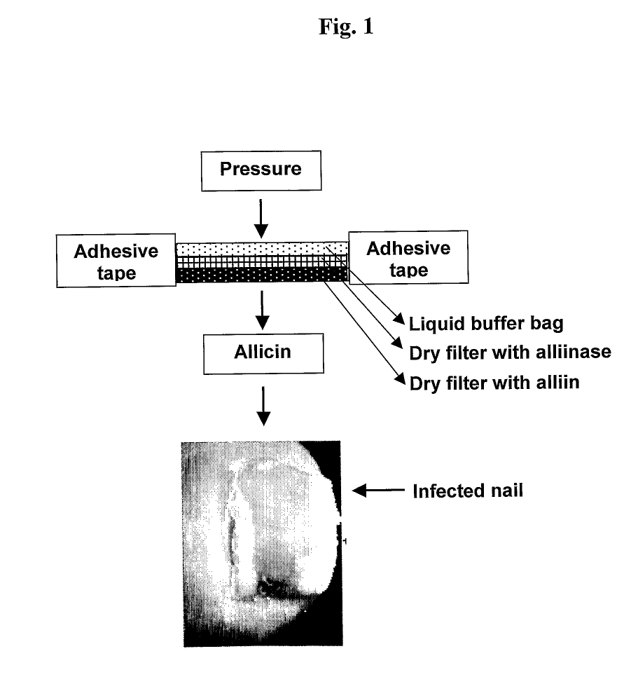

FIG. 1 shows a schematic prototype of a device according to the present invention, for topical administration of allicin to treat a fungal nail infection. In particular, the prototype designed for proof of concept and reduction to practice comprises (i) a first dry matrix containing a glass fiber filter of a certain diameter (0.5-3.0 cm), onto which a known amount of alliinase enzyme solution was adsorbed and dried by lyophilization; and (ii) a second dry matrix onto which a known amount of alliin substrate was absorbed and dried by lyophilization. The two dry filters containing the enzyme and the substrate are placed one on top of the other and can then be placed as such on the infected nail and wetted with a small amount (0.1-0.2 ml) of aqueous citrate buffer (pH 6.0, 50 mM), optionally further containing urea 1 M for enhancing the permeability through the nail, hereby referred to as the liquid solution or wetting agent. As particularly illustrated in this figure, the two dry filters are placed in an adhesive antiseptic bandage, wherein on top of this bandage, a small bag containing an aqueous buffer solution is placed. The bag containing the liquid is made of a material that is impermeable to water; however, designed to rupture and spill its content upon application of some pressure. As described above, the dry filters and the upper bag are placed in an adhesive antiseptic patch that can be placed on top of an infected nail and secured around the finger with its protruding adhesive tape. Once this adhesive bandage is well placed on the nail and secured around the finger, a moderate pressure can be applied, e.g., with a finger, on the upper bag, causing the bag to rupture and allow the liquid to wet the dry glass filter matrices below it. In a different configuration of such a device, the two dry filters are placed in an adhesive antiseptic bandage, wherein a small hole is made on top of this bandage and the liquid solution is applied with a drop bottle directly above the filters. Wetting of the filters will solubilize the alliinase and allow it to mix with the solubilized alliin adsorbed onto the matrix adjacent below, causing the biosynthesis of a measured amount of allicin, which will permeate into the infected nail below. Both in the configuration illustrated in FIG. 1, as well as in the other configuration, the allicin continuously produced on the wet matrices will permeate out of the bottom side and spread on top of the infected nail.

The spread of the allicin-containing liquid should be prevented from reaching the skin surrounding the nail. This can be accomplished by an absorbent material in a band form that the patient will place according to the nail form, around the skin borders of the nail, before applying the band with the filters. Bands containing the device of the present invention are suitable for topical antifungal application. For successful treatment, the infected nail may need to be repeatedly treated with the allicin-producing device by applying it overnight for at least two weeks, and then for about 10 weeks more once a week as a new nail begins to grow. In cases of development of a skin rash around the nail during the treatment, better isolation and protection of the skin should be secured and treatment discontinued if this situation continues.

The shapes of the present carriers may be any of various shapes commonly employed for applying to an infection site in the skin or nail. Typically, the present carriers, particularly the filters, can be shaped like a circle or semi-circle; however, they can also be cut with a scissors so as to best fit the infected nail shape.

Example 5 hereinafter shows the effective killing of both fungi and bacteria caused by allicin produced in situ following wetting of a prodrug preparation-containing carrier configured as a vaginal tampon. The list of microorganisms used in these experiments included various types of yeasts, in particular, Saccharomyces cerevisiae, Candida albicans and Candida Glabrata; several types of bacteria, in particular, group B Streptococci, methicillin-resistant Staphylococcus aureus (MRSA) and vancomycin-intermediate Staphylococcus aureus (VISA); and two strains of Trichomonas vaginalis. As shown, the LD₅₀ values measured with respect to the various microorganisms tested were in the range of 8-15 μg/ml (20-50 μM allicin).

Thus, in another embodiment, the device of the present invention comprises one solid carrier, wherein said carrier containing within the mixture of alliin and alliinase is configured as an intravaginal insertable device, or two adjacent solid carriers, wherein said carriers each containing within either the alliin or alliinase are placed one on top of the other and configured as an intravaginal insertable device. In a preferred embodiment, this device comprises one solid carrier.

The intravaginal insertable device according to the present invention can be manufactured by any suitable technology used in this field, and it may be in any suitable form such as a vaginal tampon, a vaginal sponge, a suppository, a biodegradable capsule, or a biodegradable mesh.

The device of the present invention, when comprising either one solid carrier or two adjacent solid carriers and configured as an intravaginal insertable device, may be used for treatment of bacterial or fungal infections of the vagina.

For treatment of vaginal infections, the device of the invention may be configured so as to adhere to the vaginal mucous membrane, such that contact with the vaginal lubrication provides a continuous delivery of allicin to the infected area.

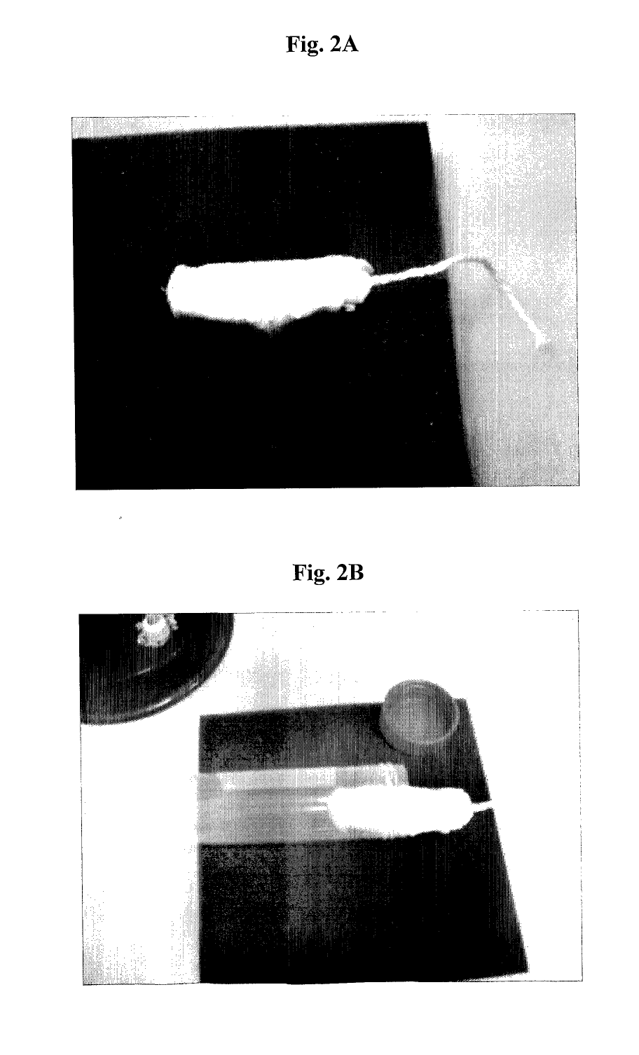

A prototype of a vaginal insertable device, in particular, a tampon, according to the present invention is shown in FIGS. 2A-2B. The vaginal insertable device contains a composition comprising a predetermined prodrug preparation, i.e., a mixture of alliin and alliinase in a stable dry form, which produces allicin in a range of about 0.1 mg to about 100 mg following insertion into the vaginal cavity and spontaneous wetting by the vaginal lubrication.

In another aspect, the present invention relates to a kit for topical administration of allicin to an infection site comprising the device of the invention and optionally a container comprising a wetting agent.

Thus, in one embodiment, the kit of the invention is used for repeated topical administration of allicin to a skin or nail infection site, and comprises a number of carriers containing alliin (marked A, for illustration), the same number of carriers containing alliinase (marked B, for illustration), and one or more containers containing the wetting agent, with instructions for placing one carrier A on top of one carrier B, or vice-versa, adhering them to the infection site, e.g., with an adhesive band, and then wetting them for continued delivery of allicin to the infection site.

In another embodiment, the kit of the invention is used for repeated topical administration of allicin to a skin or nail infection site, and comprises a number of bandages, each comprising one alliin-containing carrier on top of one alliinase-containing carrier, or vice-versa, and optionally one or more containers containing the wetting agent, with instructions for adhering said bandage to the infection site and wetting it for continued delivery of allicin to the infection site optionally by placing the container containing said wetting agent on a hole on the top of said bandage.

In a further embodiment, the kit of the invention is used for repeated topical administration of allicin to a skin or nail infection site, and comprises a number of bandages, each comprising one alliin-containing carrier on top of one alliinase-containing carrier, or vice-versa, and a small bag containing the wetting agent on top of them, with instructions for adhering said bandage to the infection site and applying moderate pressure to thereby rupture said bag and spill the wetting agent on top of said carriers for continued delivery of allicin to the infection site.

In still a further embodiment, the kit of the invention is used for repeated topical administration of allicin to a bacterial or fungal vaginal infection site, and comprises a number of carriers each containing the mixture of alliin and alliinase and configured as an intravaginal insertable device such as a tampon or a vaginal sponge, with instructions for inserting said intravaginal insertable device into the vagina, adhering it to the vaginal mucous membrane and wetting it with the vaginal lubrication for continued delivery of allicin to the infection site.

The present invention further provides a method for treating topically an infection site comprising applying to the infection site a device as defined above, optionally followed by applying a suitable amount of a wetting agent to said device, thus producing in situ allicin and continuously delivering said allicin to the infection site to thereby treat the infection.

In certain embodiments, the method of the present invention is used for treatment of bacterial or fungal skin infections, or fungal toenail or fingernail infections, in particular onychomycosis, wherein any suitable aqueous solution is used as the wetting agent.

In other embodiments, the method of the present invention is used for treatment of bacterial or fungal vaginal infections, wherein the vaginal lubrication serves, in fact, as the wetting agent.

The invention will now be illustrated by the following non-limiting Examples.

### EXAMPLES

### Example 1

### The Effect of Direct Application of Allicin on an Infected Nail

In this preliminary study, the effect of repeated topical application of pure allicin on an infected nail was examined. The study was performed on a group of 10 volunteers infected with nail fungus (onychomycosis), wherein only toenails showing visible signs of being infected with a nail fungus were chosen for the topical treatment.

In particular, a solution containing allicin (0.5 mg/ml) was prepared by passing a solution of alliin (1.5 mg/ml) through an immobilized column of alliinase as described in U.S. Pat. No. 6,689,588, and the concentration of allicin was determined by HPLC as previously described (Miron et al., 2006). The allicin solution was kept at 4° C. in citrate buffer pH 6.0 containing 1 M urea in a dark flask. A round cotton pad was cut in the approximate dimensions and size of the infected toenail (1.3 cm diameter×0.3 cm thick). 0.5 ml of the allicin solution was slowly delivered with a pipette to the surface of the cotton pad and the wet cotton pad was placed on the nail, taking precaution so as not to touch the skin surrounding the nail. The toe with the cotton pad was then covered overnight with a strip made of nylon to secure the pad on the nail and to prevent the spread of the volatile smelly odor of allicin. This treatment was repeated every night for 14 days. By the third day of the treatment, the color of the fungus in the nail turned from dark green to yellowish white, and within two weeks, the new nail that was emerging had resumed its natural color and the old part of the nail that had been infected was gradually cut off. The treatment was then repeated once a week until all the infected regions of the old nail were cut off. Two individuals did not complete the treatment, as they could not stand allicin's very typical smell of garlic, and the other eight individuals had a remarkably healthy regeneration of their toenails, wherein said toenails have remained fungus free, in some cases for over 2 years.

Allicin appears to slowly permeate the nail. Since allicin at very low concentrations is known to be very toxic against a variety of fungi, it kills the nail fungi within the nail and the new nail grows healthy.

### Example 2

### Production of Allicin by the Two-Filter System

A solution of alliin (100 μl of a solution of 50 mg/ml in water) was placed on one group of glass fiber filters; and a solution of alliinase (100 p. 1 from a solution of 120 enzyme units/ml in PBS pH 7.2 containing 5% mannitol) was placed on another group. One unit of alliinase activity is defined as the amount of enzyme converting alliin into pyruvic acid and allicin at a rate of 1 μmol/min (Miron et al., 2002). The two types of filters were exhaustively dried separately for two days in a lyophilizer. The first group of filters containing the substrate and the second group of filters containing the enzyme were then placed one on top of the other inside a small glass vial, and 1 ml of dilute (0.05 M) PBS pH 7.2 containing urea (1M) was added. Aliquots were taken after 30 minutes and analyzed by HPLC for their content of allicin.

The average amount of allicin produced by the two filters treated as described above was 0.7 mg/30 minutes, whereas no allicin was produced when filters containing either the substrate or the enzyme were wetted with buffer.

The dried filters were stored in a dry container and maintained their capacity to produce the same amount of allicin even after 40 days.

### Example 3

### Production of Allicin by the Medicated Tampon

Solutions containing either alliin or alliinase were prepared and dried in a lyophilizer as described above and the two dry components were then mixed to obtain various mixtures thereof. The rate and yield of allicin released into a neutral aqueous solution upon wetting of medicated tampons containing these mixtures were tested, and as found, the amount of allicin produced from the dry alliin/alliinase mixtures during 30 minutes at room temperature was 10 mg allicin/gram of dry powder.

In view of that, vaginal tampons in which either 0.1 or 0.25 gram of such a dry mixture, namely a prodrug preparation, has been inserted into the fissure of the cotton structure, were prepared as shown in FIG. 2A.

### Example 4

### The Antifungal Activity of the Two-Filter Delivery System

In this experiment, the effect of allicin released upon wetting of the two-filter delivery system on spores of Trichoderma hyphae was tested. In particular, four Petri dish plates containing nutrient agar for fungal growth were seeded with 5×10⁶ spores of a test soil fungi (Trichoderma hyphae) and incubated at 28° C. Growth of Trichoderma hyphae could be seen by naked eye six hours after the seeding of the spores. Two glass fiber filters, one containing dry alliin and another one containing dry alliinase, were placed on the first plate and were then wetted with a buffer as described in Example 2 hereinabove. Additional two plates in each of which a single glass fiber filter containing either the substrate or the enzyme were placed were used as controls and treated the same way. A fourth plate containing only the fungus served as the positive control, and a fifth plate without any fungus was used as the negative control.

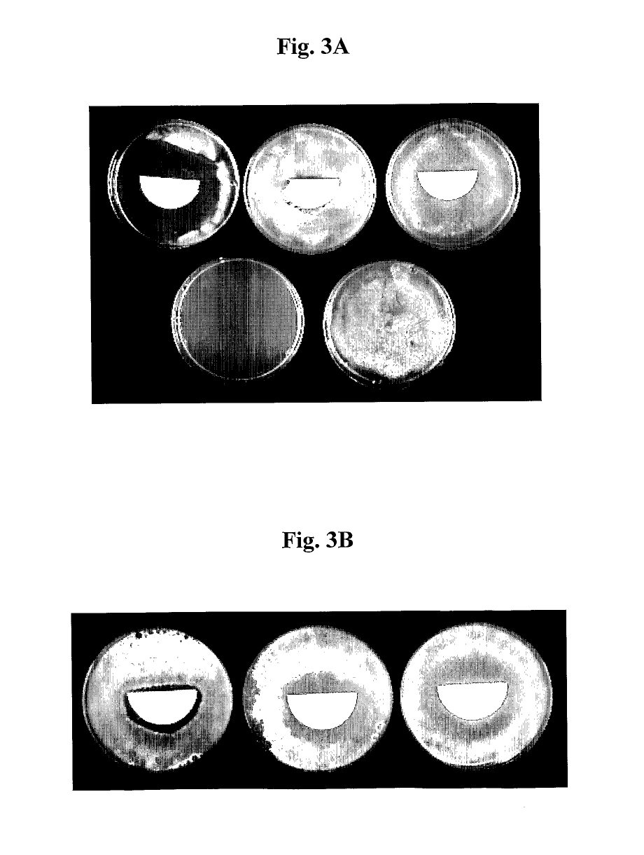

After overnight incubation, a clear halo was seen around the filter in the first Petri dish, indicating a sizable area of inhibition of fungal growth by the produced allicin, as shown in FIG. 3A. No such halos were seen in the plates in which a single filter containing either the substrate or the enzyme was placed, indicating that no allicin was produced in these cases.

A similar experiment was carried out with identical filters on Petri dish plates that contained a more mature Trichoderma hyphae growth (after 24 hours incubation from seeding of the same number of spores). The filters were placed as described above and the plates were incubated for another 16 h. As shown in FIG. 3B, in the plate in which the two filters were placed and allicin was produced, the halo of inhibition of fungal growth can be clearly seen (left plate); however, such halo is not visible in the control plates, in which a single filter containing either the substrate or the enzyme was placed (center and right plates, respectively).

### Example 5

### The Antifungal and Antibacterial Activities of the Medicated Tampon

In this experiment, the effect of allicin released upon wetting of the medicated tampons on various microorganisms, kindly provided by Dr. N. Keller from the clinical microbiology laboratory of the Sheba Medical Center (Tel HaShomer, Israel), was tested. The list of microorganisms included various types of yeasts, in particular, Saccharomyces cerevisiae, Candida albicans and Candida Glabrata; and several types of bacteria, in particular, group B Streptococci, methicillin-resistant Staphylococcus aureus (MRSA) and vancomycin-intermediate Staphylococcus aureus (VISA). In addition, two strains of Trichomonas vaginalis, one adherent to the plastic of the tube and the other non-adherent, were tested.

The effect of allicin on the different microorganisms was tested in (i) sensitivity tests to different concentrations of pure allicin in liquid culture tubes containing full nutrient medium; (ii) sensitivity tests to different amounts of allicin placed on disk filters on nutrient agar or agar/blood plates; and (iii) sensitivity tests to allicin produced from various mixtures of dry alliin and dry alliinase, both in liquid cultures and on plates as described in (ii) above. In addition, various mixtures of dry alliin and dry alliinase were placed inside the cleft of vaginal tampons to yield different amounts of allicin in tubes containing liquid medium with bacteria or yeasts, wherein following overnight incubation, aliquots were taken from the tubes and plated on nutrient agar plates, and the number of colonies developed was determined in comparison to untreated controls. The effects of allicin on Trichomonas vaginalis was tested in liquid cultures, and the viability of the trophozoites after exposure to different concentrations of allicin was determined by the methylene blue vital dye exclusion as previously described (Kiernan, 1974).

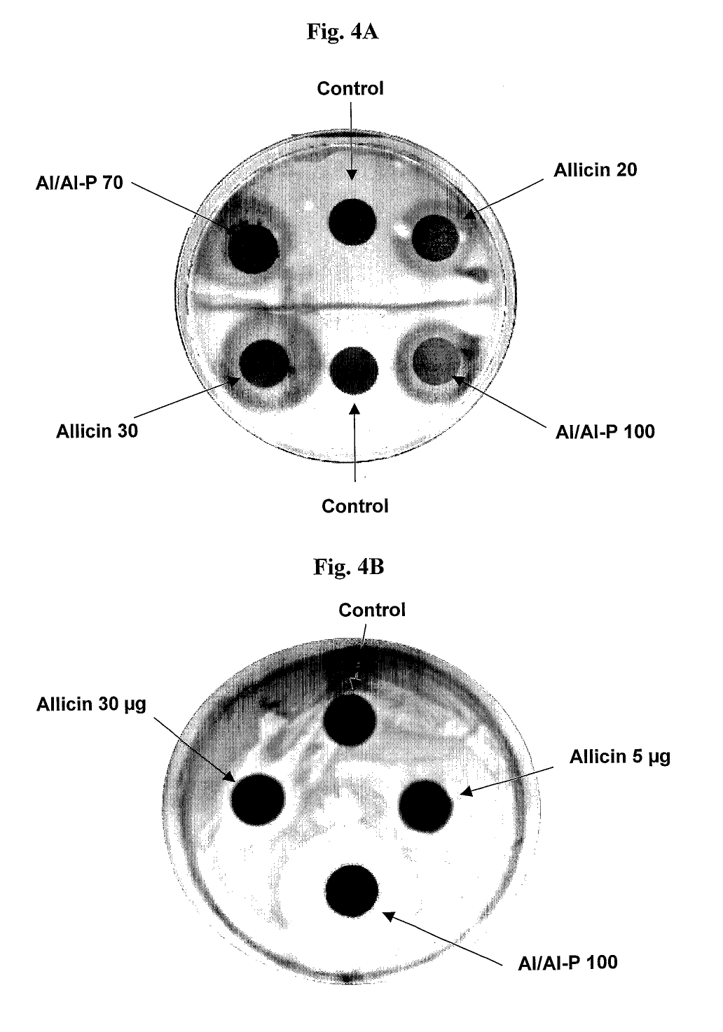

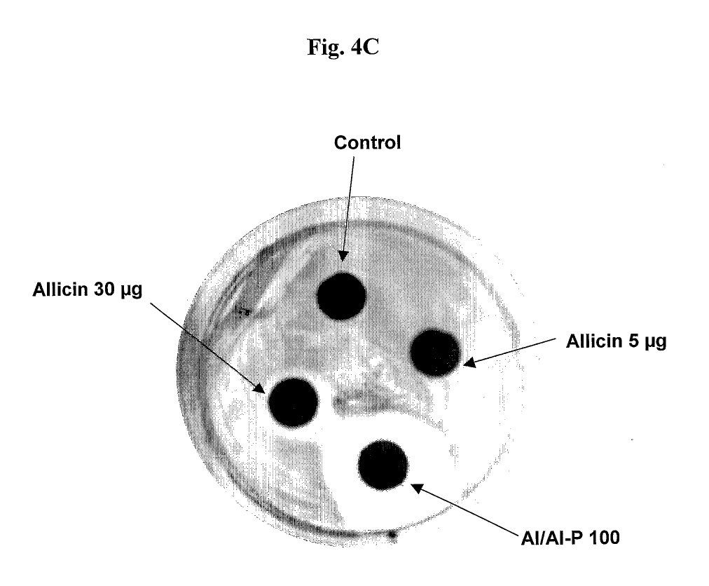

FIGS. 4A-4C show the effect of pure allicin and of mixtures of dry alliin and dry alliinase on various bacteria, in particular, group B streptococci (10⁸ bacteria) seeded on blood agar plate (4A), vancomycin-intermediate Staphylococcus aureus (VISA) seeded on regular nutrient agar plate (4B) and methicillin-resistant Staphylococcus aureus (MRSA) seeded on regular nutrient agar plate (4C), following overnight incubation at 37° C. Al/Al—P 70 and Al/Al—P 100 represent dry glass fiber filters containing a mixture of dry alliin (70 or 100 μg, respectively) and dry alliinase (2 units), which was placed on the seeded agar plate and then wetted with 100 μl water. Dry glass fiber filters on which a solution containing 5, 20 or 30 μg of pure allicin was dripped just before placing on the seeded agar served as standards, and dry glass fiber filters that were placed on the seeded agar were used as controls.

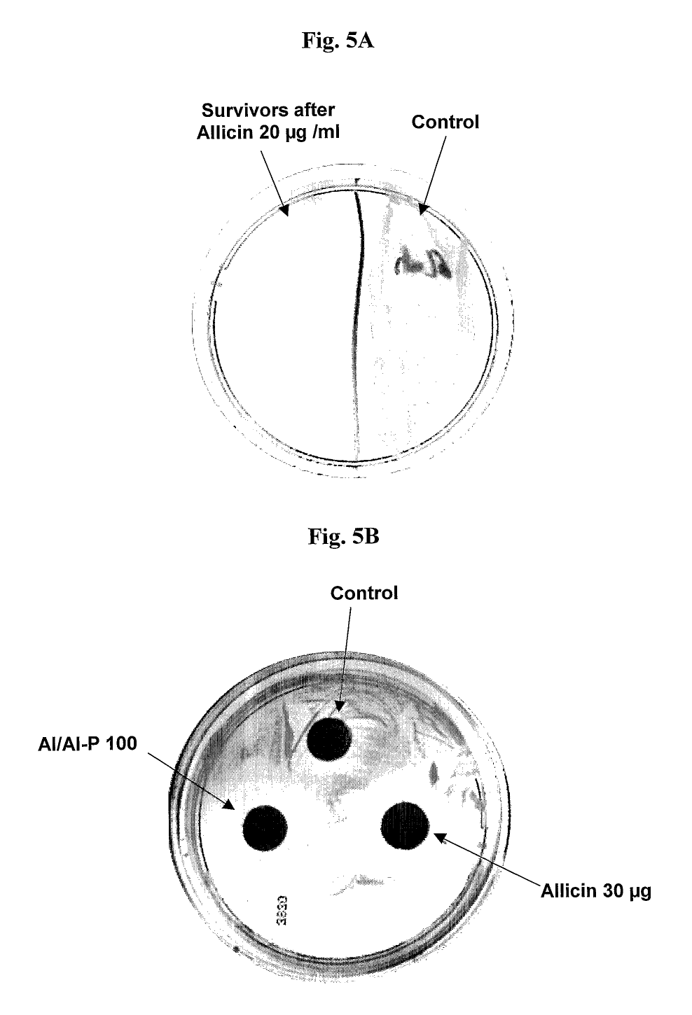

FIGS. 5A-5B show the effect of pure allicin and of a mixture of dry alliin and dry alliinase on Candida albicans (5A) and Candida glabrata (5B) seeded on agar plates, following overnight incubation at 30° C. In particular, FIG. 5A shows the effect of pure allicin (20 μg/ml) on Candida albicans, wherein in the left side of the plate, an aliquot taken from a cultivation medium of the yeast that was treated with the indicated concentration of allicin was seeded, and in the right side, an aliquot taken from an identical cultivation medium of the yeast that was not treated with allicin was seeded. FIG. 5B shows the effect of pure allicin (30 μg/ml) and of a mixture of dry alliin and dry alliinase (Al/Al—P 100) on Candida glabarta.

Table 1 hereinbelow shows the effect of different concentrations of pure allicin on the growth of Trichomonas vaginalis trophozoites. Trophozoites were grown at 37° C. in sterile tubes (4.0 ml) with Diamond's trypticase, yeast extract and bovine serum (10%) medium supplemented with a complex mixture of vitamins and cofactors, as described in Diamond et al., 1978, and were then divided into a number of tubes into which various amounts of pure allicin (5, 10, 15 and 25 μg/ml) were then added. Aliquotes were taken following incubation of 24 hours and the number of trophozoites was counted using a hemacytometer.

TABLE 1
The effect of allicin on in vitro growth of Trichomonas vaginalis

| Allicin concentration | Trophozoites (×10⁴) |
|---|---|
| 0 | 50 |
| 5 | 48 |
| 10 | 45 |
| 15 | 8 |
| 25 | 3 |

As observed from FIGS. 4-5 and Table 1, in all the experiments performed, irrespective of the type of the microorganism tested, an effective killing was caused both by pure allicin used as a standard and by the allicin produced upon wetting of the dry prodrug preparation, wherein the LD₅₀ values measured with respect to the various microorganisms tested were in the range of 8-15 μg/ml (20-50 μM allicin).

### REFERENCES

Ankri S., Mirelman D., Antimicrobial properties of allicin from garlic, Microbes and Infection, 1999, 1, 125-129
Diamond L. S., Harlow D. R., Cunnick C. C., A new medium for the axenic cultivation of Entamoeba histolytica and other Entamoeba, Trans. R. Soc. Trop. Med. Hyg., 1978, 72, 431-432
Kiernan J. A., Effects of metabolic inhibitors on vital staining with methylene blue, Histochemistry and Cell Biology, 1974, 40, 51-57
Miron T., Rabinkov A., Mirelman D., Wilchek M., Weiner L., The mode of action of Allicin: its ready permeability through phospholipid membranes may contribute to its biological activity, Biochem. Biophys. Acta, 2000, 1463, 20-30
Miron T., Shin I., Feigenblat G., Weiner L., Mirelman D., Wilchek M., Rabinkov A., A spectrophotometric assay for allicin, alliin, and alliinase (alliin lyase) with a chromogenic thiol: reaction of 4-mercaptopyridine with thiosulfinates, Anal. Biochem., 2002, 307, 76-83
Miron T., SivaRaman H., Rabinkov A., Mirelman D., Wilchek M., A method for continuous production of allicin using immobilized alliinase, nal. Biochem., 2006, 351, 152-154
Prager-Khoutorsky M., Goncharov I., Rabinkov A., Mirelman D., Geiger B., Bershadsky A., Allicin inhibits cell polarization, migration and division via its direct effect on microtubules, Cell Motil. Cytoskeleton, 2007, 64, 321-337
Rabinkov A., Xiao-zhu Z., Grafi G., Galili G., Mirelman D., Alliin lyase (alliinase) from garlic (allium sativum): biochemical characterization and cDNA cloning, Appl. Biochem. Biotechnol., 1994, 48, 149-171
Shadkchan Y., Shemesh E., Mirelman D., Miron T., Rabinkov A., Wilchek M., Osherov N., Efficacy of Allicin, the reactive molecule of garlic, in inhibiting Aspergillus spp. in vitro, and in a murine model of disseminated aspergillosis, J. Antimicrob. Chemother., 2004, 53, 832-836

1. A device for topical administration of allicin to an infection site, comprising either one solid carrier or two adjacent solid carriers, dry alliin and dry alliinase, wherein either a mixture of said dry alliin and dry alliinase is contained within said one solid carrier or dry alliin and dry alliinase are each separately contained within each one of said two adjacent solid carriers, whereby in contact with the infection site and a wetting agent, the alliinase acts on the alliin and allicin is produced in situ and administered to the infection site.

2. The device of claim 1, wherein said carriers are selected from glass fiber filter, cotton, gauze or a polysaccharide-based polymer absorbent material such as starch and cellulose.

3. The device of claim 1, wherein said wetting agent is a buffer with a pH range of 6-7.5, such as a citrate buffer or phosphate-buffered saline, optionally further comprising a permeability enhancer such as urea; or said wetting agent is a bodily fluid such as vaginal lubrication or saliva.

4. The device of claim 1, comprising one solid carrier placed in a bandage or two adjacent solid carriers placed one on top of the other in a bandage.

5. The device of claim 4, wherein said bandage is an adhesive band.

6. The device of claim 4, comprising a hole on the top of said bandage for placing a small drop bottle with the wetting agent; or a small bag containing the wetting agent on top of said one solid carrier or two adjacent solid carriers.

7. The device of claim 6, wherein said bag is made of a water impermeable material designed for rupturing under moderate pressure, thus spilling the wetting agent on top said one solid carrier or two adjacent solid carriers.

8. The device of claim 4, for treatment of a bacterial or fungal skin infection; or a fungal toenail or fingernail infection.

9. The device of claim 8, wherein said fungal toenail infection is onychomycosis.

10. The device of claim 1, comprising one solid carrier configured as an intravaginal insertable device or two adjacent solid carriers placed one on top of the other and configured as an intravaginal insertable device.

11. The device of claim 10, for treatment of a bacterial or fungal vaginal infection.

12. A kit for topical administration of allicin to an infection site comprising the device of claim 1 and optionally a container comprising the wetting agent.

13. The kit of claim 12 for repeated topical administration of allicin to a skin or nail infection site, comprising:
(i) a number of carriers containing alliin, the same number of carriers containing alliinase, and one or more containers containing the wetting agent, with instructions for placing one alliin-containing carrier on top of one alliinase-containing carrier, or vice-versa, adhering them to the infection site, and then wetting them for continued delivery of allicin to the infection site;
(ii) a number of bandages, each comprising one alliin-containing carrier on top of one alliinase-containing carrier, or vice-versa, and optionally one or more containers containing the wetting agent, with instructions for adhering said bandage to the infection site and wetting it for continued delivery of allicin to the infection site optionally by placing the container containing said wetting agent on a hole on the top of said bandage; or
(iii) a number of bandages, each comprising one alliin-containing carrier on top of one alliinase-containing carrier, or vice-versa, and a small bag containing the wetting agent on top of them, with instructions for adhering said bandage to the infection site and applying moderate pressure to thereby rupture said bag and spill the wetting agent on top of said carriers for continued delivery of allicin to the infection site.

14. The kit of claim 12 for repeated topical administration of allicin to a bacterial or fungal vaginal infection site, comprising a number of carriers each containing the mixture of alliin and alliinase and configured as an intravaginal insertable device, with instructions for inserting said intravaginal insertable device into the vagina, adhering it to the vaginal mucous membrane and wetting it with the vaginal lubrication for continued delivery of allicin to the infection site.

15. A method for treating topically an infection site comprising applying to the infection site the device of claim 1, optionally followed by applying a suitable amount of a wetting agent to said device, thus producing in situ allicin and continuously delivering said allicin to the infection site to thereby treat the infection.

16. The method of claim 15, for treatment of a bacterial or fungal skin infection; a fungal toenail or fingernail infection; or a bacterial or fungal vaginal infection.

17. The device of claim 2, wherein said carriers are glass fiber filter or cotton.

18. The device of claim 4, comprising two adjacent solid carriers placed one on top of the other in a bandage.

19. The device of claim 10, comprising one solid carrier configured as an intravaginal insertable device.
